(19) United States
(12) Patent Application Publication
(10) Pub. No.: US 2022/0251053 A1
(43) Pub. Date: Aug. 11, 2022

(54) PROCESS OF PREPARING 2-(PHENYLIMINO)-1,3-THIAZOLIDIN-4-ONES

(71) Applicant: Bayer Aktiengesellschaft, Leverkusen (DE)

(72) Inventors: Thomas HIMMLER, Odenthal (DE); Julia Johanna HAHN, Duesseldorf (DE)

(21) Appl. No.: 17/625,273

(22) PCT Filed: Jul. 8, 2020
(86) PCT No.: PCT/EP2020/069172

§ 371 Date: Jan. 6, 2022

(30) Foreign Application Priority Data
Jul. 10, 2019 (EP) 19185391.0

(51) Int. Cl. C07D 277/38

(52) U.S. Cl. CPC C07D 277/38

(57) ABSTRACT

The present invention relates to a method for preparing 2-(phenylimino)-1,3-thiazolidin-4-ones of the general formula (I).

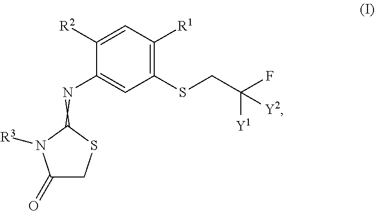

in which Y¹, Y², R¹, R² and R³ are as defined in the description.

The present invention relates to a method for preparing 2-(phenylimino)-1,3-thiazolidin-4-ones of the general formula (I).

2-(Phenylimino)-1,3-thiazolidin-4-ones and corresponding derivatives are of great importance in the pharmaceutical and agrochemical industry as intermediates in the production of, for example, chiral sulfoxides. Sulfoxides of this kind are used for example in crop protection as acaricides (see e.g. WO2013/092350 or WO2015/150348).

The chemical synthesis of 2-(phenylimino)-1,3-thiazolidin-4-ones is known. This can be accomplished, for example, by reacting an appropriately substituted thiourea of the general formula (II) with an acetic acid derivative of the general formula (III) (see e.g. WO2013/092350; EP 985670; Advances in Heterocycl. Chem. 25, (1979) 85). There are in principle a number of methods for preparing the thiourea of the general formula (II). A simple and effective method consists of the reaction of an appropriately substituted aniline of the general formula (IV) with an isothiocyanate of the general formula (V) (WO2014/202510). Conversely, it is also possible to obtain the thiourea of the general formula (II) by reacting an aryl isothiocyanate of the general formula (VI) with an amine of the general formula (VII) (JP2011/042611).

Thus, a familiar method of preparing 2-(phenylimino)-1,3-thiazolidin-4-ones of the general formula (I) is characterized in that, in a first step, an aniline of the general formula (IV) is reacted with an isothiocyanate of the general formula (V), or an aryl isothiocyanate of the general formula (VI) is reacted with an amine of the general formula (VII), and the thiourea of the general formula (II) thereby formed is then isolated, for example by filtration. In a second step of the known method, the thiourea of the general formula (II) is then reacted with an acetic acid derivative of the general formula (III) in the presence of a base to form the 2-(phenylimino)-1,3-thiazolidin-4-one of the general formula (I).

A disadvantage of this method is the laborious procedure involving two separate steps with the isolation of the thiourea intermediate. This is time-consuming and incurs high costs. In addition, depending on the nature of the diluent used, it can result in precipitates of the thiourea of the general formula (II) that can be so voluminous that the reaction mixture becomes impossible to stir and cannot be discharged from the reaction vessel. If this occurs, isolation of the thiourea intermediate becomes practically impossible. Moreover, when subjected to thermal stress, as can also occur for example when drying a solid after filtration, thioureas are known (Synthesis 1984, 825-7; WO2014/189753; J Labelled Comp. and Radiopharmaceuticals 22(1985) 313-27) to undergo partial cleavage back to the starting compounds (thermal instability).

The method (A) known from the prior art is shown in scheme (1), in which X, Y¹, Y², W, R¹, R² and R³ are as defined below.

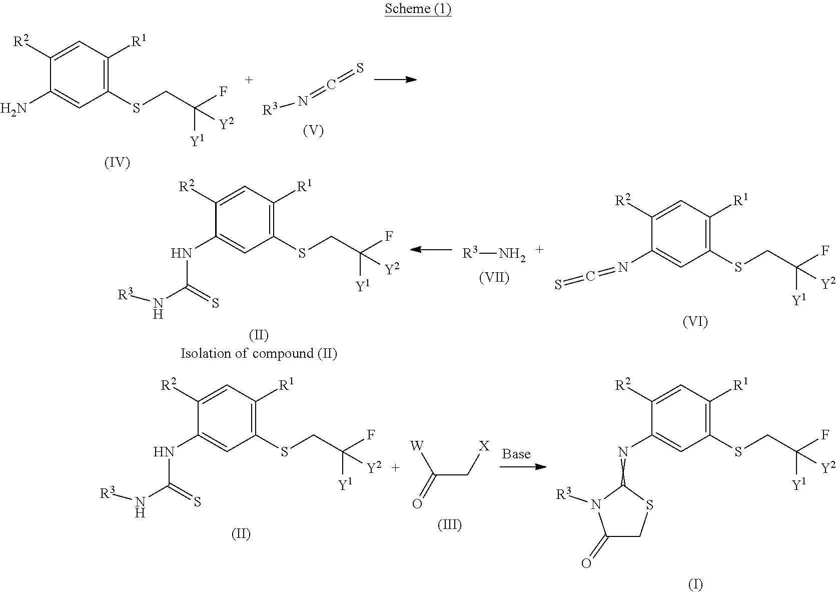

In view of the disadvantages outlined above, there is an urgent need for a simplified, industrially and economically practicable method for preparing 2-(phenylimino)-1,3-thiazolidin-4-ones of the general formula (I). The 2-(phenylimino)-1,3-thiazolidin-4-ones obtainable with such a method should preferably be afforded in high yield and high purity. In particular, the method that is sought should allow the desired target compounds to be obtained without the need for complex methods of isolation. In addition, the method that is sought should shorten the reaction time appreciably and preferably permit the use of diluents suitable for use on an industrial scale.

It was surprisingly found that 2-(phenylimino)-1,3-thiazolidin-4-ones of the general formula (I) can be prepared by reacting an aryl isothiocyanate of the general formula (VI) with an amine of the general formula (VII) in the presence of an acetic acid derivative of the general formula (III) and a base, with the thiourea of the general formula (II) that is formed as an intermediate reacting directly and preferably in situ to form the 2-(phenylimino)-1,3-thiazolidin-4-one.

The present invention provides a method for preparing 2-(phenylimino)-1,3-thiazolidin-4-ones of the general formula (I)

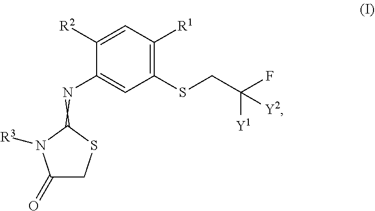

in which
Y¹ and Y² are independently fluorine, chlorine or hydrogen,
R¹ and R² are independently hydrogen, C₁-C₁₂ alkyl, C₁-C₁₂ haloalkyl, cyano, halogen or nitro, and
R³ is optionally substituted C₆-C₁₀ aryl, C₁-C₁₂ alkyl or C₁-C₁₂ haloalkyl, in which the substituents are selected from halogen, C₁-C₆ alkyl, C₃-C₁₀ cycloalkyl, cyano, nitro, hydroxy, C₁-C₆ alkoxy, C₁-C₆ haloalkyl and C₁-C₆ haloalkoxy, in particular from fluorine, chlorine, C₁-C₃ alkyl, C₃-C₆ cycloalkyl, cyclopropyl, cyano, C₁-C₃ alkoxy, C₁-C₃ haloalkyl and C₁-C₃ haloalkoxy,
characterized in that an aryl isothiocyanate of the formula (VI):

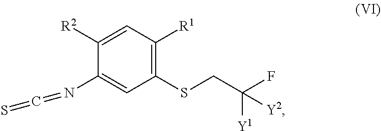

in which Y¹, Y², R¹ and R² are as defined above,
in the presence of an acetic acid derivative of the formula (III)

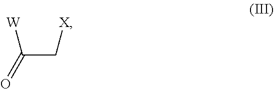

in which
X is bromine, chlorine, OSO₂Me, OSO₂Ph, OSO₂(4-Me-Ph) or OSO₂CF₃, and
W is OH or an O(C₁-C₆ alkyl) radical,
and in the presence of a base, reacts with an amine of the formula (VII)

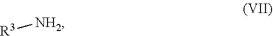

in which
R³ is as defined above,
initially to form the thiourea of the formula (II)

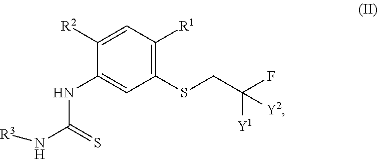

in which Y¹, Y², R¹, R² and R³ are as defined above,
which is then converted into the compound of the formula (I), with the acetic acid derivative of the formula (III) being initially present in the reaction mixture prior to the addition to the reaction mixture of at least one of the compounds of the formulas (VI) and (VII).

The acetic acid derivative of the formula (III) is therefore already present when the aryl isothiocyanate of the formula (VI) reacts with the amine of the formula (VII) to form the thiourea of the formula (II). It has no adverse effect on this reaction; on the contrary, it ensures that—rather than accumulating in the reaction mixture—the thiourea of the formula (II) is immediately further converted into the compound of the formula (I).

In other words, the thiourea of the formula (II) is immediately converted in situ into the compound of the formula (I), i.e. the thiourea of the formula (II) formed as an intermediate undergoes an immediate further reaction in situ to form the 2-(phenylimino)-1,3-thiazolidin-4-one of the formula (I).

The compounds of the formula (I) may be present as the E- or Z-isomer or as a mixture of these isomers. This is indicated by the crossed double bond in the formula (I). In an individual embodiment of the invention, the compound is in each case in the form of the E-isomer. In another individual embodiment of the invention, the compound is in each case in the form of the Z-isomer. In another individual embodiment of the invention, the compound is in the form of a mixture of the E- and Z-isomers. In a preferred individual embodiment of the invention, the compound is in the form of the Z-isomer or a mixture of the E- and Z-isomers in which the proportion of the Z-isomer is greater than 50% and with increasing preference greater than 60%, 65%, 70%, 75%, 80%, 85%, 90%, 95%, based on the total amount of the E- and Z-isomers in the mixture.

Preferred, particularly preferred and very particularly preferred definitions of the radicals X, Y¹, Y², W, R¹, R² and R³ listed in the formulas (I), (II), (III), (VI) and (VII) mentioned above are elucidated below.

It is preferable when

X is bromine or chlorine,
Y¹ and Y² are independently fluorine, chlorine or hydrogen,
W is an O(C₁-C₆ alkyl) radical,
R¹ and R² are independently fluorine, chlorine, C₁-C₃ alkyl or hydrogen and
R³ is optionally substituted phenyl, C₁-C₆ alkyl or C₁-C₆ haloalkyl, in which the substituents are selected from halogen, C₁-C₆ alkyl, C₃-C₁₀ cycloalkyl, cyano, nitro, hydroxy, C₁-C₆ alkoxy, C₁-C₆ haloalkyl and C₁-C₆ haloalkoxy, in particular from fluorine, chlorine, C₁-C₃ alkyl, C₃-C₆ cycloalkyl, cyclopropyl, cyano, C₁-C₃ alkoxy, C₁-C₃ haloalkyl and C₁-C₃ haloalkoxy.

It is particularly preferable when

X is bromine or chlorine,
Y¹ and Y² are independently fluorine or hydrogen,
W is an O(C₁-C₆ alkyl) radical,
R¹ and R² are independently fluorine, chlorine, hydrogen or methyl and
R³ is C₁-C₆ alkyl or C₁-C₆ haloalkyl.

It is very particularly preferable when

X is bromine or chlorine,
Y¹ and Y² are fluorine,
W is an OCH₃ or OC₂H₅ radical,
R¹ and R² are independently fluorine, hydrogen or methyl and
R³ is C₁-C₆ haloalkyl.

It is most preferable when

X is bromine or chlorine,
Y¹ and Y² are fluorine,

### W is OCH₃,

R¹ is methyl,
R² is fluorine and
R³ is CH₂CF₃.

Surprisingly, the 2-(phenylimino)-1,3-thiazolidin-4-ones of the formula (I) can be prepared by the method of the invention with good yields and in high purity. The acetic acid derivative of the formula (III) has little or no adverse effect on the reaction of the compounds of the formulae (VI) and (VII) to form the thiourea of the formula (II) and results, since it is already present when the thiourea of the formula (II) is formed, in the immediate further conversion of the latter into the compound of the formula (I). This avoids the formation of a sticky, pasty reaction mixture that is difficult to handle. It was in no way foreseeable that the acetic acid derivative of the formula (III) could therefore be added to the reaction mixture at an early stage and thus be immediately available for the reaction of the thiourea of the formula (II). This accordingly brings improvements both in the purity and yield of the target compound of the formula (I) and, importantly, in process economics, particularly on an industrial scale. Moreover, the method of the invention allows the use of diluents that are suitable for industrial-scale production, in particular ones in which voluminous precipitates of the thioureas of the formula (II) can otherwise occur. A further advantage for process economics brought by the method of the invention is that it allows the desired target compounds to be obtained without the need for complex isolation procedures for the intermediate.

The method of the invention can be elucidated on the basis of the following scheme (2), in which X, Y¹, Y², W, R¹, R² and R³ are as defined above. Scheme (2) illustrates the clean conversion. As described, the compound of the formula (III) is present in the reaction mixture prior to the addition to the reaction mixture of at least one of the compounds of the formulas (VI) and (VII).

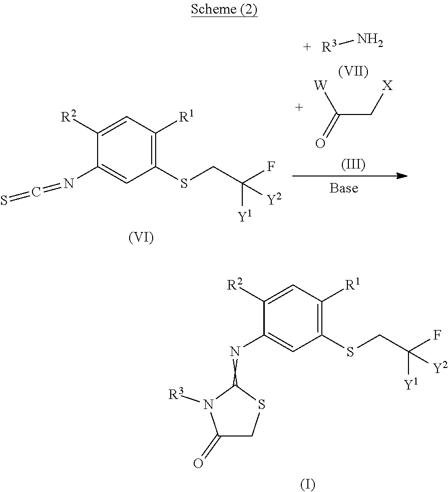

### General Definitions

In the context of the present invention, the term “halogens” (Hal) encompasses, unless otherwise defined, elements selected from the group consisting of fluorine, chlorine, bromine and iodine, preference being given to using fluorine, chlorine and bromine, and particular preference to using fluorine and chlorine.

Optionally substituted groups may be singly or multiply substituted; if multiply substituted, the substituents may be identical or different. Unless otherwise stated at the relevant position, substituents are selected from halogen, C₁-C₆ alkyl, C₃-C₁₀ cycloalkyl, cyano, nitro, hydroxy, C₁-C₆ alkoxy, C₁-C₆ haloalkyl and C₁-C₆ haloalkoxy, in particular from fluorine, chlorine, C₁-C₃ alkyl, C₃-C₆ cycloalkyl, cyclopropyl, cyano, C₁-C₃ alkoxy, C₁-C₃ haloalkyl and C₁-C₃ haloalkoxy.

Alkyl groups substituted by one or more halogen atoms (Hal) are, for example, selected from trifluoromethyl (CF₃), difluoromethyl (CHF₂), CF₃CH₂, ClCH₂ or CF₃CCl₂.

Alkyl groups in the context of the present invention are, unless otherwise defined, linear, branched or cyclic saturated hydrocarbon groups.

The definition C₁-C₁₂ alkyl encompasses the widest range defined herein for an alkyl group. Specifically, this definition encompasses, for example, methyl, ethyl, n-propyl, isopropyl, n-butyl, isobutyl, sec-butyl and t-butyl, n-pentyl, n-hexyl, 1,3-dimethylbutyl, 3,3-dimethylbutyl, n-heptyl, n-nonyl, n-decyl, n-undecyl, n-dodecyl.

Aryl groups in the context of the present invention are, unless otherwise defined, aromatic hydrocarbon groups, which may include zero, one, two or more heteroatoms (selected from O, N, P and S).

Specifically, this definition encompasses, for example, cyclopentadienyl, phenyl, cycloheptatrienyl, cyclooctatetraenyl, naphthyl and anthracenyl; 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl; 1-pyrrolyl, 1-pyrazolyl, 1,2,4-triazol-1-yl, 1-imidazolyl, 1,2,3-triazol-1-yl, 1,3,4-triazol-1-yl; 3-pyridazinyl, 4-pyridazinyl, 2-pyrimidinyl, 4-pyrimidinyl, 5-pyrimidinyl, 2-pyrazinyl, 1,3,5-triazin-2-yl and 1,2,4-triazin-3-yl.

The conversion of the aryl isothiocyanate of the formula (VI) into the compound having the formula (I) is preferably carried out in the presence of a diluent. Suitable diluents in the method of the invention are in particular the following: tetrahydrofuran (THF), dioxane, diethyl ether, methyl tert-butyl ether (MTBE), tert-amyl methyl ether (TAME), 2-methyl-THF, acetonitrile (ACN), acetone, butyronitrile, ethyl acetate, isopropyl acetate, butyl acetate, pentyl acetate, methyl isobutyl ketone, ethylene carbonate, propylene carbonate, N,N-dimethylacetamide (DMAc), N,N-dimethylformamide (DMF), N-methylpyrrolidone, dimethyl sulfoxide (DMSO), sulfolane, tetrachloroethylene, tetrachloroethane, dichloropropane, methylene chloride (dichloromethane, DCM), dichlorobutane, chloroform, carbon tetrachloride, trichloroethane, trichloroethylene, pentachloroethane, 1,2-dichloroethane, toluene, ortho-xylene, meta-xylene, para-xylene, ethylbenzene, mesitylene, chlorobenzene, 1,2-dichlorobenzene, anisole, n-pentane, n-hexane, n-heptane, n-octane, 1,2,4-trimethylpentane (isooctane), petroleum ether 40/55, special boiling point spirit 80/110, cyclohexane or methylcyclohexane. Mixtures of said diluents may also be used.

Preferred diluents in the method of the invention are methylene chloride, chloroform, 1,2-dichloroethane, acetonitrile, acetone, ethyl acetate, methyl tert-butyl ether (MTBE), tetrahydrofuran (THF), 2-methyl-THF, N,N-dimethylacetamide (DMAc), N,N-dimethylformamide (DMF), toluene, ortho-xylene, meta-xylene, para-xylene, ethylbenzene, mesitylene, chlorobenzene, 1,2-dichlorobenzene, anisole, n-heptane, n-octane, 1,2,4-trimethylpentane (isooctane), petroleum ether 40/55, special boiling point spirit 80/110, methylcyclohexane or mixtures of said diluents.

Particularly preferred diluents are acetonitrile, ethyl acetate, tetrahydrofuran (THF), toluene, ortho-xylene, meta-xylene, para-xylene, ethylbenzene, mesitylene, chlorobenzene, 1,2-dichlorobenzene, anisole, n-heptane, 1,2,4-trimethylpentane (isooctane), petroleum ether 40/55, special boiling point spirit 80/110, methylcyclohexane or mixtures of said diluents. Very particular preference is given to toluene, ortho-xylene, meta-xylene, para-xylene, ethylbenzene or chlorobenzene or mixtures of said diluents.

The amine of the formula (VII) is preferably used in a molar ratio from 0.95:1 to 2:1 based on the aryl isothiocyanate of the formula (VI). Further preference is given to molar ratios from 1.01:1 to 1.5:1, again in each case based on the aryl isothiocyanate of the formula (VI).

The base used in the method of the invention may be an organic or an inorganic base. Examples of organic bases are trimethylamine, triethylamine, tributylamine and ethyldiisopropylamine. Examples of inorganic bases are potassium acetate, sodium acetate, lithium hydroxide, potassium hydroxide, sodium hydroxide, potassium hydrogen carbonate, sodium hydrogen carbonate, potassium carbonate, sodium carbonate, caesium carbonate, calcium carbonate and magnesium carbonate. Preference is given to potassium hydroxide, sodium hydroxide, potassium carbonate and sodium carbonate. Particular preference is given to potassium carbonate.

In the method of the invention, the base is preferably used in a molar ratio from 0.8:1 to 3:1 based on the aryl isothiocyanate of the formula (VI). Further preference is given to molar ratios from 1:1 to 2:1, again in each case based on the aryl isothiocyanate of the formula (VI).

In the method of the invention, the acetic acid derivative of the formula (III) is preferably used in a molar ratio from 0.9 to 2 based on the aryl isothiocyanate of the formula (VI). Further preference is given to molar ratios from 1.0 to 1.5, again in each case based on the aryl isothiocyanate of the formula (VI).

The method of the invention is generally carried out at a temperature between −20° C. and 150° C., preferably between 0° C. and 120° C., most preferably between 5° C. and 80° C.

The reaction is typically carried out at standard pressure, but may also be carried out at elevated or reduced pressure.

The desired compounds of the formula (I) may be isolated for example by subsequent filtration or extraction. Such processes are known to those skilled in the art.

The present invention is elucidated in detail by the examples that follow, although the examples should not be interpreted in such a manner that they restrict the invention.

### EXAMPLE

### Example 1: Synthesis of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one in Toluene

A reaction vessel was charged with 10 ml of toluene, 1.216 g [4.32 mmol] of 1-fluoro-2-isothiocyanato-5-methyl-4-[(2,2,2-trifluoroethyl)sulfanyl]benzene, 0.841 g [5.5 mmol] of methyl bromoacetate and 0.967 g [7.5 mmol] of potassium carbonate. 0.743 g [7.5 mmol] of 2,2,2-trifluoroethanamine was added dropwise, with stirring, and stirring at 20-25° C. was then continued for 24 hours. The reaction mixture was a very readily stirrable, thin suspension throughout this time. This was cooled to room temperature, diluted with 10 ml of toluene, and stirred with 15 ml of water, after which the phases were separated, the aqueous phase was extracted with 10 ml of toluene, the combined organic phases were washed with 10 ml of 1 N hydrochloric acid, drying was carried out over sodium sulfate and the organic phase was concentrated. This afforded 1.93 g of product having a purity by HPLC of 90.7%, corresponding to a yield of 96% of theory.

### COMPARATIVE EXAMPLES

### Comparative Example 1: Synthesis of 1-{2-fluoro-4-methyl-5-[(2,2,2-trifluoroethyl)sulfanyl]phenyl}-3-(2,2,2-trifluoroethyl)thiourea in Toluene

5.0 g of 2-fluoro-4-methyl-5-[(2,2,2-trifluoroethyl)sulfanyl]aniline [20.9 mmol, 1.0 equiv.] was added to 30 ml of toluene and to this was added dropwise, at room temperature, 3.2 g of 1,1,1-trifluoro-2-isothiocyanatoethane [23.0 mmol, 1.1 equiv.]. The reaction mixture was stirred at room temperature for 3 hours, resulting in the formation from the original solution of a very thick, poorly stirrable suspension. Monitoring of the reaction indicated only about 85% conversion. The reaction mixture was heated to 50° C. in order to make it partially stirrable again. After 3 hours at 50° C., complete conversion still had not been achieved, consequently the reaction mixture was heated to 70° C. Complete conversion was still not achieved even after 3 hours at 70° C. (HPLC monitoring of the reaction indicated that 0.9% of the aniline was still present). The reaction mixture was cooled to 5° C. and the very thick, pasty suspension transferred to a suction filter as thoroughly as possible and the solid isolated. The solid obtained was washed with cold MTBE and dried under reduced pressure. This afforded 5.1 g of the target product as a beige solid (61% of theory). Concentration of the filtrate gave a further 2.2 g of a brown solid, which had a target product content of approx. 60% (17% of theory). The poor isolated yield is due in part also to the relatively large losses during transfer of the very thick suspension to the suction filter.

### Comparative Example 2: Synthesis of 1-{2-fluoro-4-methyl-5-[(2,2,2-trifluoroethyl)sulfanyl]phenyl}-3-(2,2,2-trifluoroethyl)thiourea in Methylcyclohexane

A reaction vessel was charged with 77 ml of methylcyclohexane (MCH) and 11.9 g [50 mmol] of 2-fluoro-4-methyl-5-[(2,2,2-trifluoroethyl)sulfanyl]aniline. This was heated to 50° C. and 8.1 g [57.5 mmol] of 1,1,1-trifluoro-2-isothiocyanatoethane was added dropwise at this temperature, with stirring, over a period of approx. 5 minutes. After a few minutes the target product began to precipitate out, causing the reaction mixture to become a thick, unstirrable paste. Even the addition of a further 80 ml of methylcyclohexane did not make the mixture stirrable again. The reaction mixture was cooled to 20° C. and rinsed out of the reaction vessel with large amounts of MCH. The solid was filtered off with suction, washed with MCH and dried. This afforded 18.55 g of product having a purity by HPLC analysis of 98.5% (a/a), corresponding to a yield of 96% of theory. Thus, although the yield is very good, the extremely pasty consistency of the reaction mixture makes the methodology unworkable on an industrial scale.

### Comparative Example 3: Synthesis of (2Z)-2-({2-fluoro-4-methyl-5-[(2,2,2-trifluoroethyl)sulfanyl]phenyl}imino)-3-(2,2,2-trifluoroethyl)-1,3-thiazolidin-4-one in Toluene

7.1 g of 1,1,1-trifluoro-2-isothiocyanatoethane [95%, 48.0 mmol, 1.2 equiv.] was dissolved in 40 ml of toluene and stirred (400 rpm) with 9.57 g of 2-fluoro-4-methyl-5-[(2,2,2-trifluoroethyl)sulfanyl]aniline (40.0 mmol, 1.1 equiv.) for 30 min at 20° C., resulting in the formation from the yellowish solution of a suspension containing a white solid. After 1 hour the suspension was no longer stirrable, but monitoring of the reaction by HPLC analyses of the suspension indicated only about 65% conversion. A further 10 ml of toluene was added, the stirring speed was increased to 600 rpm and the reaction mixture was heated to 40° C., as a result of which the mixture became moderately stirrable again. After 3 hours at 40° C. (HPLC monitoring of the reaction showed approx. 87% conversion), 8.3 g of solid potassium carbonate [60.0 mmol, 1.5 equiv.] was added. After a further 30 min, 8.0 g of methyl 2-bromoacetate [52.0 mmol, 1.3 equiv.] was added at 40° C. over a period of 1 hour and the reaction mixture was stirred at 40° C. for 20 hours, resulting in the formation of a suspension of potassium bromide and potassium carbonate in a toluene solution of the target product that was once again readily stirrable. HPLC monitoring of the reaction at this point showed complete conversion of the aniline and only traces of the intermediate thiourea. The reaction mixture was cooled to 20° C., stirred at 20° C. for a further 17 hours and filtered. The solid was washed with a little toluene and the combined filtrates concentrated to 66.8 g of a reddish brown toluene solution, which was shown by HPLC against an external standard to contain 21.10% of the target product (84% of theory) and neither aniline nor the thiourea intermediate.

1. A method for preparing 2-(phenylimino)-1,3-thiazolidin-4-ones of formula (I) 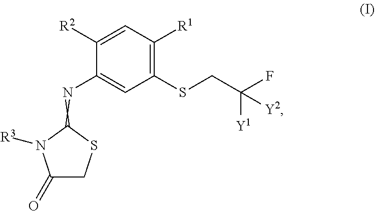
in which
Y¹ and Y² are independently fluorine, chlorine or hydrogen,
R¹ and R² are independently hydrogen, C₁-C₁₂ alkyl, C₁-C₁₂ haloalkyl, cyano, halogen or nitro, and
R³ is optionally substituted C₆-C₁₀ aryl, C₁-C₁₂ alkyl or C₁-C₁₂ haloalkyl, in which the substituents are selected from halogen, C₁-C₆ alkyl, C₃-C₁₀ cycloalkyl, cyano, nitro, hydroxy, C₁-C₆ alkoxy, C₁-C₆ haloalkyl and C₁-C₆ haloalkoxy,
wherein an aryl isothiocyanate of formula (VI) 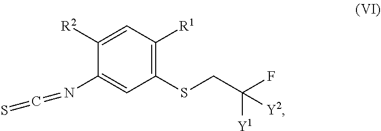
in which Y¹, Y², R¹ and R² are as defined above,
comprising reacting, in the presence of an acetic acid derivative of formula (III) 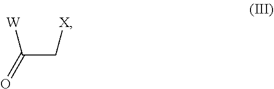
in which
X is bromine, chlorine, OSO₂Me, OSO₂Ph, OSO₂(4-Me-Ph) or OSO₂CF₃, and
W is OH or an O(C₁-C₆ alkyl) radical,
and in the presence of a base,
with an amine of formula (VII) 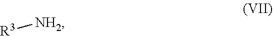
in which
R³ is as defined above,
initially to form the thiourea of formula (II) 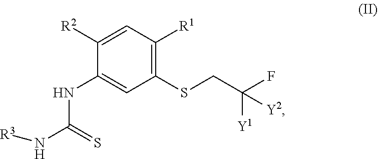
in which Y¹, Y², R¹, R² and R³ are as defined above,
which is then converted into the compound of formula (I), with the acetic acid derivative of formula (III) being initially present in the reaction mixture prior to the addition to the reaction mixture of at least one of the compounds of formulas (VI) and (VII).

2. The method according to claim 1, wherein the compound of formula (I) is in the form of the Z-isomer or a mixture of the E- and Z-isomers in which the proportion of the Z-isomer is greater than 50% based on the total amount of the E- and Z-isomers in the mixture.

3. The method according to claim 1, wherein
X is bromine or chlorine,
Y¹ and Y² are independently fluorine, chlorine or hydrogen,
W is an O(C₁-C₆ alkyl) radical,
R¹ and R² are independently fluorine, chlorine, C₁-C₃ alkyl or hydrogen and
R³ is optionally substituted phenyl, C₁-C₆ alkyl or C₁-C₆ haloalkyl, in which the substituents are selected from halogen, C₁-C₆ alkyl, C₃-C₁₀ cycloalkyl, cyano, nitro, hydroxy, C₁-C₆ alkoxy, C₁-C₆ haloalkyl and C₁-C₆ haloalkoxy.

4. The method according to claim 1, wherein
X is bromine or chlorine,
Y¹ and Y² are independently fluorine or hydrogen,
W is an O(C₁-C₆ alkyl) radical,
R¹ and R² are independently fluorine, chlorine, hydrogen or methyl and
R³ is C₁-C₆ alkyl or C₁-C₆ haloalkyl.

5. The method according to claim 1, wherein
X is bromine or chlorine,
Y¹ and Y² are fluorine,
W is an OCH₃ or OC₂H₅ radical,
R¹ and R² are independently fluorine, hydrogen or methyl and
R³ is C₁-C₆ haloalkyl.

6. The method according to claim 1, wherein
X is bromine or chlorine,
Y¹ and Y² are fluorine,
W is OCH₃,
R¹ is methyl,
R² is fluorine and
R³ is CH₂CF₃.

7. The method according to claim 1, wherein conversion of the aryl isothiocyanate of formula (VI) into the compound of formula (I) takes place in the presence of a diluent selected from tetrahydrofuran (THF), dioxane, diethyl ether, methyl tert-butyl ether (MTBE), tert-amyl methyl ether (TAME), 2-methyl-THF, acetonitrile (ACN), acetone, butyronitrile, ethyl acetate, isopropyl acetate, butyl acetate, pentyl acetate, methyl isobutyl ketone, ethylene carbonate, propylene carbonate, N,N-dimethylacetamide (DMAc), N,N-dimethylformamide (DMF), N-methylpyrrolidone, dimethyl sulfoxide (DMSO), sulfolane, tetrachloroethylene, tetrachloroethane, dichloropropane, methylene chloride (dichloromethane, DCM), dichlorobutane, chloroform, carbon tetrachloride, trichloroethane, trichloroethylene, pentachloroethane, 1,2-dichloroethane, toluene, ortho-xylene, meta-xylene, para-xylene, ethylbenzene, mesitylene, chlorobenzene, 1,2-dichlorobenzene, anisole, n-pentane, n-hexane, n-heptane, n-octane, 1,2,4-trimethylpentane (isooctane), petroleum ether 40/55, special boiling point spirit 80/110, cyclohexane or methylcyclohexane and mixtures thereof.

8. The method according to claim 1, wherein the amine of formula (VII) is present in a molar ratio from 0.95:1 to 2:1 based on the aryl isothiocyanate of formula (VI).

9. The method according to claim 1, wherein the base is an organic base selected from trimethylamine, triethylamine, tributylamine and ethyldiisopropylamine, or that the base is an inorganic base selected from potassium acetate, sodium acetate, lithium hydroxide, potassium hydroxide, sodium hydroxide, potassium hydrogen carbonate, sodium hydrogen carbonate, potassium carbonate, sodium carbonate, caesium carbonate, calcium carbonate and magnesium carbonate.

10. The method according to claim 1, wherein the base is used in a molar ratio from 0.8:1 to 3:1 based on the aryl isothiocyanate of formula (VI).

11. The method according to claim 1, wherein the acetic acid derivative of formula (III) is present in a molar ratio from 0.9:1 to 2:1 based on the aryl isothiocyanate of formula (VI).

12. The method according to claim 7, wherein the diluent is selected from toluene. ortho-xylene, meta-xylene, para-xylene, ethylbenzene, chlorobenzene and a mixture of said diluents and/or the base potassium carbonate.

13. The method according to claim 1, wherein said method is carried out at a temperature between −20 and 150° C.
